(19) United States
(12) Patent Application Publication
(10) Pub. No.: US 2014/0275876 A1
(43) Pub. Date: Sep. 18, 2014

(54) SYSTEMS AND METHODS FOR LOCATING AND/OR IDENTIFYING A WIRELESS SENSOR ASSOCIATED WITH A PATIENT MONITOR

(71) Applicant: COVIDIEN LP, Mansfield, MA (US)

(72) Inventors: Bryan Hansen, Mead, CO (US); Niranjan Maharajh, Broomfield, CO (US); Alissa Wong, Niwot, CO (US)

(21) Appl. No.: 13/841,286

(22) Filed: Mar. 15, 2013

(51) Int. Cl. A61B 5/00; A61B 5/1455

(52) U.S. Cl. CPC A61B 5/0022; A61B 5/14551; A61B 5/742
USPC 600/323; 600/300

(57) ABSTRACT

The present embodiments include one or more wireless patient sensors that may be coupled, or recoupled, to a patient monitor to ensure that the patient monitor is displaying information from the intended source. In other embodiments, the wireless sensors described herein may use one or more unique tokens to establish a coupling between the wireless sensor and the patient monitor though wireless communication channels. In addition, the present embodiments relate to wireless sensors and patient monitors each having one or more audio/visual features that indicate the coupling status between them, and may also locate and/or identify one or more wireless sensors coupled to the patient monitor, or vise versa. The audio/visual features may include an audio feature, a visual feature, or a combination thereof. In particular, the visual feature may be a display feature (e.g., electronic ink display).

### BACKGROUND

The present disclosure relates generally to patient monitoring systems and, more particularly, to locating and/or identifying one or more patient sensors associated with one or more patient monitors.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for non-invasively monitoring many such physiological characteristics. These devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient.

Such patient sensors may communicate with a patient monitor using a communication cable. For example, a patient sensor may use such a communication cable to send a signal, corresponding to a measurement performed by the sensor, to the patient monitor for processing. However, the use of communication cables may limit the range of applications available, as the cables may become prohibitively expensive at long distances as well as limit a patient's range of motion by physically tethering the patient to a monitoring device. As such, it may be desirable to monitor the physiological parameters of a patient with wireless sensors. Indeed, certain monitors, such as pulse oximetry monitors, may be equipped with features (e.g., wireless communication technologies) that enable a patient to freely move about while remote monitoring is being performed.

Wireless sensors are typically paired with a patient monitor to ensure that the patient monitor is displaying information from the intended source. However, a permanent factory configured pairing creates a situation where a particular sensor can only be used with a particular patient monitor. Moreover, with multiple wireless sensors in use within a facility, an operator may be unsure which wireless sensor is sending a signal, corresponding to a physiological measurement, to the patient monitor. As such, it may be difficult to safely and accurately identify the patient being monitored when viewing physiological information on a patient monitor. Additionally, while wireless sensors enable remote monitoring of a patient, there is often a need for human involvement in immediate assessment of the measured physiological parameters of the patient, even when the patient monitor is not locally available.

### BRIEF DESCRIPTION OF THE DRAWINGS

Advantages of the disclosed techniques may become apparent upon reading the following detailed description and upon reference to the drawings in which:

FIG. 1 is a perspective view of a patient monitoring system having a patient monitor and a wireless patient sensor, in accordance with an embodiment;

FIG. 2 is a block diagram of the wireless patient sensor of FIG. 1, illustrating a plurality of components that may be present within the body of the wireless patient sensor;

FIG. 3 illustrates an embodiment of the patient monitoring system having the wireless module of the patient monitor coupled to the wireless module of the wireless sensor, and having audio/visual features;

FIG. 4 depicts a process flow diagram of an embodiment of a method for identifying or locating the patient monitor or the wireless sensor with the audio/visual features;

FIG. 5 illustrates an embodiment of the patient monitoring system having the wireless module of the patient monitor coupled to the wireless module of the wireless sensor through a pairing feature;

FIG. 6 depicts a process flow diagram of an embodiment of a method for coupling (i.e., pairing) one or more wireless sensors to the patient monitor with the pairing feature;

FIG. 7 illustrates an embodiment of the patient monitoring system having the wireless module of the patient monitor coupled to the wireless module of the wireless sensor through one or more unique tokens;

FIG. 8 illustrates an embodiment of the token having one or more hole patterns 152 representative of the unique identifier;

FIG. 9 illustrates an embodiment of the token having 2D or 3D quick response (QR) codes representative of the unique identifier;

FIG. 10 illustrates an embodiment of the token having bar codes representative of the unique identifier;

FIG. 11 depicts a process flow diagram of an embodiment of a method for coupling (i.e., pairing) one or more wireless sensors to the patient monitor with the one or more unique tokens;

FIG. 12 illustrates an embodiment of the visual feature (i.e., a visual display) on the wireless sensor that can be performed in conjunction with or independently of the systems and methods of FIGS. 3-11; and

FIG. 13 depicts a process flow diagram of an embodiment of a method for displaying on a visual feature (e.g., electronic ink display) patient-specific physiological information and/or patient identification information transferred from a transmitting device to the wireless sensors.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Wireless patient sensors may be used to provide a patient with a greater freedom of movement when compared to wired patient sensors. In certain circumstances, it may be desirable to have a wireless sensor and a patient monitor functioning together to monitor one or more physiological parameters of the patient. However, a permanent factory pairing between a wireless sensor and a patient monitor limits the versatility of wireless sensors. Moreover, with multiple wireless sensors in use within a facility, an operator may be unsure which wireless sensor is sending a signal to the patient monitor. Indeed, an operator may find it difficult to safely and accurately identify the patient being remotely monitoring when viewing physiological information on a patient monitor. In addition, it may be desirable to assess the physiological information of a patient being remotely monitored, even when a patient monitor is not locally available. As such, it some situations, it may be desirable to couple, or recouple, a wireless sensor with a patient monitor to ensure that the patient monitor is displaying information from the intended source and to provide coupling flexibility to wireless sensors as they move about a facility. In some situations, it may be beneficial to provide an audio or visual feature on the wireless sensor or the patient monitor as an indicator of the coupling between the sensor and the monitor. In yet other situations, it may be desirable to provide a display feature on the wireless sensor that displays the identification information (e.g., of a patient, a patient monitor, a wireless sensor, or a combination thereof), or the measured physiological parameters for remote assessment by an operator (i.e., when a patient monitor is not locally available).

With the forgoing in mind, the present embodiments relate to wireless patient sensors that may be coupled, or recoupled, to a patient monitor to ensure that the patient monitor is displaying information from the intended source. For example, in certain embodiments, the wireless sensors described herein may incorporate one or more physical pairing features (e.g., electrical features, polarized magnets, etc.) for coupling the wireless sensor to the patient monitor through wireless communication channels. In other embodiments, the wireless sensors described herein may use one or more unique tokens to establish a coupling between the wireless sensor and the patient monitor though wireless communication channels. In such embodiments, each unique token may be any card, paper, or plastic that has a unique identifying feature that identifies the wireless sensor and the patient monitor to be coupled. In particular, an operator may be alerted when the wireless sensor and the patient monitor are coupled, if they are unable to be coupled, or if wireless connectivity is lost.

In addition, the present embodiments relate to wireless sensors and patient monitors each having one or more audio/visual features that indicate the coupling status between them. In certain embodiments, the audio/visual features may alert an operator when a wireless sensor and a patient monitor are coupled, are unable to be coupled, or when wireless connectivity is lost. In other embodiments, the audio/visual features may indicate or identify one or more wireless sensors coupled to the patient monitor, or vise versa. For example, the audio/visual feature on a wireless sensor may audibly or visually indicate or identify the patient being remotely monitored when it is activated or triggered by an activation feature on the patient monitor. The audio/visual features may include an audio feature, a visual feature, or a combination thereof. For example, the wireless sensors or patient monitors described herein may incorporate one or more audio features, such as an alarm indicator, voice warnings, and so forth. As a further example, the wireless sensors and patient monitors described herein may also be equipped with visual indicators, such as flashing lights, color changing bands, visual displays, and so forth. In particular, the visual feature may be a display feature (e.g., electronic ink display or E-ink display) incorporated into the wireless sensors described herein, and may provide identification information and/or measured physiological parameter information for a patient. In certain embodiments, the electronic ink display may be a paper-like display that provides a high contrast, flicker-free display that is also relatively thin and flexible, allowing it be incorporated on a wireless sensor. Again, the identification information on the display feature may indicate the coupling status of the wireless sensors and patient monitors, while the measured physiological parameter information may enable an operator to remotely assess the patient's condition.

One embodiment of a patient monitoring system 10 that may benefit from the approaches described herein is depicted in FIG. 1. The illustrated patient monitoring system 10 includes a patient monitor 12 and a wireless patient sensor 14. The patient monitoring system 10 is configured to enable the calculation of one or more physiological parameters of a patient by either of the wireless patient sensor 14 or the patient monitor 12. For example, in one embodiment, the patient monitoring system 10 may be configured to enable the wireless patient sensor 14 to perform various calculations in order to limit wireless communication and to conserve battery power in the wireless patient sensor 14. Although the illustrated embodiment of system 10 is a pulse oximetry monitoring system, it should be noted that the patent monitoring system 10 may be configured to perform any number of measurements on a patient to determine one or more physiological parameters of the patient. That is, while the pulse oximetry monitoring system 10 may determine pulse rates and blood oxygen saturation levels (e.g., SpO₂ values) for a patient, the system 10 may, additionally or alternatively, be configured to determine a patient's respiration rate, glucose levels, hemoglobin levels, hematocrit levels, tissue hydration, patient temperature, cardiogram information, blood pressure, or pulse transit time, as well as other physiological parameters. Furthermore, while the illustrated embodiment includes the wireless patient sensor 14 that communicates with the patient monitor 12 in a wireless fashion, the present approaches are also applicable to configurations in which a patient sensor communicates with the patient monitor 12 using a wired connection. Such communication may be carried out using light guides, one or more conductors (e.g., via a cable), or any other suitable communication and/or power transmission features. In such embodiments, either or both of the patient monitor 12 and wireless patient sensor 14 may perform any of the determinations or calculations described herein.

The patient monitor 12 may include a display 16, a wireless module 18 (e.g., transceiver including a transmitter and/or receiver) for transmitting and receiving wireless data, a memory, a processor, and various monitoring and control features. Based on data received from the wireless patient sensor 14, the patient monitor 12 may display physiological parameters of the patient on display 16. The system 10 may also be communicatively coupled to a multi-parameter monitor 20 to facilitate presentation of patient data, such as pulse oximetry data determined by system 10 and/or physiological parameters determined by other patient monitoring systems (e.g., electrocardiographic (ECG) monitoring system, a respiration monitoring system, a blood pressure monitoring system, etc.). For example, the multi-parameter monitor 20 may display a graph of SpO₂ values, a current pulse rate, a graph of blood pressure readings, an electrocardiograph, and/or other related patient data in a centralized location for quick reference by a medical professional. In some embodiments, the patient monitor 12 may include an original equipment manufacturer (OEM) pulse oximetry module. The pulse oximetry module may calibrate a sensor, reduce noise in signals received from a sensor, extract signals representing arterial signals, and process these signals into physiological information such as pulse rate and peripheral oxygen saturation (SpO₂).

Like the patient monitor 12, the patient sensor 14 also includes a wireless module 22 (e.g., transceiver including a transmitter and/or receiver). The wireless module 22 of the sensor 14 may establish wireless communication with the wireless module 18 of the patient monitor 12 using any suitable protocol. For example, the wireless modules 18, 22 may be capable of communicating using the IEEE 802.15.4 standard, and may be, for example, ZigBee, WirelessHART, or MiWi modules. Additionally or alternatively, the wireless modules 18, 22 may be capable of communicating using the Bluetooth standard, one or more of the IEEE 802.11 standards, an ultra-wideband (UWB) standard, or a near-field communication (NFC) standard. In certain embodiments, the wireless module 22 of the patient sensor 14 may be used to transmit either raw detector signals or calculated physiological parameter values to the patient monitor 12 depending on the noise level and/or complexity of the detector signal. In certain embodiments, the wireless module 22 on the wireless sensor 14 may be coupled, or recoupled, to the wireless module 18 on the patient monitor 12 by establishing, or reestablishing, wireless communication with the wireless module 18. In other embodiments, the wireless sensors 14 described herein may incorporate one or more physical pairing features (e.g., polarized magnets embedded into the wireless sensor 14) for coupling the wireless sensor 14 to the patient monitor 12. In yet other embodiments, as described below, the wireless sensors 14 may use one or more unique tokens to establish a coupling (e.g., operatively and/or communicatively coupling) between the wireless sensor 14 and the patient monitor 12. The unique token may be any card, paper, or plastic that has a unique identifying feature (e.g., a RFID tag, a 3D QR code, a 2D QR code, a unique hole pattern, a unique barcode) that identifies the wireless sensor and the patient monitor to be coupled In particular, an operator may be alerted when the wireless sensor 14 and the patient monitor 12 are coupled, if they are unable to be coupled, or if wireless connectivity is lost.

The illustrated wireless patient sensor 14 includes an emitter 24 and a detector 26 coupled to a body 28 of the sensor 14. The body 28 of the wireless patient sensor 14 facilitates attachment to a patient tissue (e.g., a patient's finger, ear, forehead, or toe). For example, in the illustrated embodiment, the sensor 14 is configured to attach to a finger of a patient 30. When attached to a pulsatile tissue such as the finger, the emitter 24 may transmit light at certain wavelengths (e.g., for example, red light and/or infrared light) into the tissue, wherein the red light may have a wavelength of about 600 to 700 nm, and the IR light may have a wavelength of about 800 to 1000 nm. The detector 26 may receive the red and IR light after it has passed through or is reflected by the tissue. The emitter 24 may emit the light using one, two, or more LEDs, or other suitable light sources. The detector 26 may be any suitable light detecting feature, such as a photodiode or photo-detector. The process of emission and detection of the light after passing through or reflection by the tissue is used to characterize the nature of the underlying tissue, as the amount of light that passes through the patient tissue and other characteristics of the light may vary according to the changing amount of certain blood constituents in the tissue. In addition to the emission and detection features noted above, the wireless patient sensor 14 may include a button or switch 32, which may be used for other features related to the embodiments described herein.

FIG. 2 is a block diagram of an embodiment of the wireless medical sensor system 10 that may be configured to implement the techniques described herein. By way of example, embodiments of the system 10 may be implemented with any suitable medical sensor and patient monitor, such as those available from Nellcor Puritan Bennett LLC. The system 10 may include the patient monitor 12 and the sensor 14, which may be configured to obtain, for example, a plethysmographic signal from patient tissue at certain predetermined wavelengths. The photoplethysmographic sensor 14 may be communicatively connected to the patient monitor 12 via wireless communication 20 (shown in FIG. 1). When the system 10 is operating, light from the emitter 28 may pass into the patient 36 and be scattered and detected by the detector 30. The sensor 14 may include a microprocessor 38 connected to a bus 40. Also connected to the bus 40 may be a RAM memory 42 and an optional ROM memory 44. A time processing unit (TPU) 46 may provide timing control signals to light drive circuitry 48 which may control when the emitter 28 is illuminated, and if multiple light sources are used, the multiplexed timing for the different light sources. The TPU 46 may optionally also control the gating-in of signals from the detector 30 through an amplifier 50 and a switching circuit 52. These signals may be sampled at the proper time, depending upon which of multiple light sources is illuminated, if multiple light sources are used. The received signal from the detector 30 may be passed through an amplifier 54, a low pass filter 56, and an analog-to-digital converter 58.

The digital data may then be stored in a queued serial module (QSM) 60, for later downloading to the RAM 42 as the QSM 60 fills up. Alternatively, the processor 38 may read the A/D converter after each sample, without the use of QSM 60. In one embodiment, there may be multiple parallel paths of separate amplifier, filter and A/D converters for multiple light wavelengths or spectra received. This raw digital data may be further processed by the wireless medical sensor 14 into specific data of interest, such as pulse rate, blood oxygen saturation, and so forth. The data of interest may take up significantly less storage space than the raw data.

For example, a raw 16-bit digital stream of photoplethysmographic data of between approximately 50 Hz or less to 2000 Hz or more (e.g., approximately 1211 Hz) may be sampled down to between approximately 10 Hz to 200 Hz (e.g., approximately 57.5 Hz), before being processed to obtain an instantaneous pulse rate at a given time, which may take up only approximately 8 bits.

In an embodiment, the sensor 14 may also contain an encoder 62 that provides signals indicative of the wavelength of one or more light sources of the emitter 28, which may allow for selection of appropriate calibration coefficients for calculating a physiological parameter such as blood oxygen saturation. The encoder 62 may, for instance, be a coded resistor, EEPROM or other coding devices (such as a capacitor, inductor, PROM, RFID, parallel resonant circuits, or a colorimetric indicator) that may provide a signal to the processor 38 related to the characteristics of the photoplethysmographic sensor 14 that may allow the processor 38 to determine the appropriate calibration characteristics for the photoplethysmographic sensor 14. Further, the encoder 62 may include encryption coding that prevents a disposable part of the photoplethysmographic sensor 14 from being recognized by a processor 38 that is not able to decode the encryption. For example, a detector/decoder 64 may be required to translate information from the encoder 62 before it can be properly handled by the processor 38. In some embodiments, the encoder 62 and/or the detector/decoder 64 may not be present. Additionally or alternatively, the processor 38 may encode processed sensor data before transmission of the data to the patient monitor 12.

In various embodiments, based at least in part upon the value of the received signals corresponding to the light received by detector 30, the microprocessor 38 may calculate a physiological parameter of interest using various algorithms. These algorithms may utilize coefficients, which may be empirically determined, corresponding to, for example, the wavelengths of light used. These may be stored in the ROM 44 or in other nonvolatile memory 66 including flash or One-Time Programmable (OTP) memory. In a two-wavelength system, the particular set of coefficients chosen for any pair of wavelength spectra may be determined by the value indicated by the encoder 62 corresponding to a particular light source provided by the emitter 28. For example, the first wavelength may be a wavelength that is highly sensitive to small quantities of deoxyhemoglobin in blood, and the second wavelength may be a complimentary wavelength. Specifically, for example, such wavelengths may be produced by orange, red, infrared, green, and/or yellow LEDs. Different wavelengths may be selected based on instructions from the patient monitor 12, based preferences stored in a nonvolatile storage 66, or depending on whether the button or switch 34 has been selected, as determined by the button or switch decoder 68 or automatically based on an algorithm executed by the processor 38. The instructions from the patient monitor 12 may be transmitted wirelessly to the sensor 14 in the manner described below with reference to FIGS. 3-6, and may be selected at the patient monitor 12 by a switch on the patient monitor 12, a keyboard, or a port providing instructions from a remote host computer.

Nonvolatile memory 66 may store caregiver preferences, patient information, or various parameters, discussed below, which may be used in the operation of the sensor 14. Software for performing the configuration of the sensor 14 and for carrying out the techniques described herein may also be stored on the nonvolatile memory 66, or may be stored on the ROM 44. The nonvolatile memory 66 and/or RAM 42 may also store historical values of various discrete medical data points. By way of example, the nonvolatile memory 66 and/or RAM 42 may store values of instantaneous pulse rate for every second or every heart beat of the most recent five minutes. These stored values may be used as factors in determining the wireless data update rate, as discussed in greater detail below.

A battery 70 may supply the wireless medical sensor 14 with operating power. By way of example, the battery 70 may be a rechargeable battery, such as a lithium ion or lithium polymer battery, or may be a single-use battery such as an alkaline or lithium battery. Due to the techniques described herein to reduce battery consumption, the battery 70 may be of a much lower capacity, and accordingly much smaller and/or cheaper, than a battery needed to power a similar wireless sensor that does not employ these techniques. A battery meter 72 may provide the expected remaining power of the battery 70 to the microprocessor 38. The remaining battery life indicated by the battery meter 72 may be used as a factor in determining the wireless data update rate, as discussed in greater detail below. The wireless medical sensor 14 may also include a movement sensor 74 that may sense when the patient 36 moves the sensor 14. The movement sensor 74 may include, for example, a digital accelerometer that may indicate a state of motion of the patient 36. Whether the patient is at rest or moving, as indicated by the movement sensor 74, may also be used as a factor in determining the wireless data update rate, as discussed in greater detail below. The microprocessor 38 may carry out these techniques based on instructions stored in the RAM 42, the ROM 44, the nonvolatile memory 66, or based on instructions received from the patient monitor 12.

In particular, the wireless module 22 in the sensor 14 may be wirelessly (e.g., operatively and/or communicatively) coupled to the wireless module 18 in the patient monitor 12. In certain embodiments, the wireless sensor 14 and the patient monitor 12 each have one or more audio/visual features and one or more activation features, as described below with respect to FIGS. 3-13, that indicate the coupling status between them. In certain embodiments, the audio/visual features and the activation features may be operatively coupled to the processor(s) 38, the wireless modules 18, 22, and/or other components within the system as described in FIG. 2. These features may alert an operator when a wireless sensor and a patient monitor are coupled, are unable to be coupled, or when wireless connectivity is lost. In other embodiments, the audio/visual features may indicate or identify one or more wireless sensors coupled to the patient monitor, or vise versa, as described below with respect to FIGS. 3-6.

As noted above, the wireless module 18 of the monitor 12 and the wireless module 22 of the sensor 14 may be coupled or paired, as discussed with respect to FIGS. 1 and 2. Indeed, the present embodiments provide various methods for pairing the monitor 12 with the sensor 14 in accordance with the embodiments discussed above. For example, FIGS. 3 and 4 illustrate the wireless modules 18, 20 coupled using one of any number of wireless communication methods described above. FIGS. 5 and 6 illustrate the wireless modules 18, 20 coupled using one or more physical pairing features on the monitor 12 and the sensor 14. FIGS. 7-11 illustrate systems and method for a unique token that is used to couple the sensor 14 to the patient monitor 12. In particular, FIGS. 3-13 each illustrate an audio/visual feature (e.g., an audio feature, a visual feature, or a combination thereof) that can be triggered by an activation feature to indicate the coupling status of the monitor 12 and the sensor 14, or to locate and/or identify the monitor 12 and the sensor 14 that are coupled. FIGS. 12-13 each illustrate an embodiment of the visual feature on the sensor 14 (i.e., visual E-Ink display) that can be performed in conjunction with or independently of the systems and methods of FIGS. 3-11.

Referring now to FIG. 3, an embodiment of the patient monitoring system 10 is illustrated having the wireless module 18 of the patient monitor 12 coupled to the wireless module 22 of the wireless sensor 14. The depicted embodiment illustrates an audio/visual feature 80 and an activation feature 82 on the wireless sensor 14, and an audio/visual feature 81 and an activation feature 83 on the patient monitor 12. In certain embodiments, the audio/visual feature 80, 81 includes an audio feature 84 and a visual feature 86. In particular, the audio/visual feature 80, 81 is triggered by the activation feature 82, 83, and may be use to indicate the coupling status of the wireless sensor 14 and the patient monitor 12. For example, as the wireless sensor 14 establishes wireless connectivity to the patient monitor 12, the audio/visual feature 81 on the patient monitor 12 and the audio/visual feature 80 on the wireless sensor 14 may indicate the status of connectivity (e.g., wireless communication established, wireless communication cannot be established, or wireless communication is establishing). As a further example, an operator may engage the activation feature 83 on the patient monitor 12 to trigger the audio/visual feature 80 on the wireless sensor 14. The triggered audio/visual feature 80 on the wireless sensor 14 may be used to locate and/or identify the patient sensor 14 wirelessly communicating with the patient monitor 12.

As shown, the wireless module 18 of the patient monitor 12 establishes an identification channel 90 and a communication channel 92 with the wireless module 22 of the wireless sensor 14. As noted above, the wireless module 22 of the sensor 14 may establish the wireless communication (e.g., via identification channel 90 and/or communication channel 92) with the wireless module 18 of the patient monitor 12 using any suitable protocol. In certain embodiments, the identification channel 90 may couple (e.g., pair) the patient monitor 12 with the wireless sensor 14. For example, the identification channel 90 may be used to exchange identification information for the patient 30, identification information for the patient monitor 12, identification information for the wireless sensor 14, or a combination thereof. In addition, the identification channel 90 may provide a communication pathway for the activation features 82, 83 to interact with the audio/visual features 80, 81. In other embodiments, the communication channel 92 may be used to exchange patient-specific data, such as physiological parameter trend data acquired over time for a particular patient 30. For example, the wireless sensor 14 may be attached to the finger of the patient 30, and may be configured to store patient-specific data (e.g., physiologic trend data acquired over time for a particular patient) in the access memory (RAM) 60 and/or non-volatile (NV) memory 62. Such data may also be transferred to the patient monitor 12 through the communication channel 92. In particular, in certain embodiments, the communication channel 92 is established only after safely and securely establishing wireless communication through the identification channel 90. In other embodiments, wireless communication between the sensor 14 and the monitor 12 may occur through a single channel that may exchange both identification information and/or patient-specific data.

More specifically, the identification channel 90 may exchange identification information between the wireless sensor 14 and the patient monitor 12 for the patient 30, for the patient monitor 12, or for the wireless sensor 14. Identification information for the patient 30 may include any information used to uniquely identify a patient 30 within a facility, such as, for example, a patient ID number, a patient name, a unique bar code number, a unique serial number, a patient identification tag or bracelet, or a combination thereof. In addition, identification information for the sensor 14 or the monitor 12 may be any information that can be used to identify the name, type, or location of the sensor 14 or the monitor 12, such as, for example, a unique serial number, a unique identification code, a barcode, a RFID tag, or any unique identification information provided by a manufacturing entity. In certain embodiments, wireless communication (e.g., identification channel 90) may be established when the wireless sensor 14 is attached to the finger of the patient 30, and the switch 32 is used to activate (e.g., turn on the emitter 24 and detector 26) the sensor 14. After establishing wireless communication with a locally available patient monitor 12, in certain embodiments, it may be desirable to couple (e.g., pair) the wireless sensor 14 and the patient monitor 12 through the identification channel 90 by exchanging unique identification information such as those provided above.

In particular, the activation features 82, 83 and the audio/visual features 80, 81 on the sensor 14 and the monitor 12 may use the identification channel 90 to communicate with one another. The activation features 82, 83 may be any selectable input or feature that the operator can interact with on either the monitor 12 or the sensor 14, such as a button or a switch. In other embodiments, the size, shape, locations, and/or labels for the activation features 82, 83 can vary. In particular, the activation features 82, 83 may be engaged to change information shown on the display 16, or on the visual display on the sensor 14 (as described in FIGS. 13-16).

The audio/visual features 80, 81 on either the monitor 12 or the sensor 14 may include an audio feature 84 and a visual feature 86. In certain embodiments, the audio feature 84 (e.g., audio indicator 84) may be any indicator type that audibly facilitates the identification of the audio/visual features 80, 81 on the patient monitor 12 or on the wireless sensor 14 by the operator or patient 30. For example, the audio feature 84 may be a speaker for emitting audible indications (e.g., alarms or beep tones), possibly with various frequencies, pitches, and/or volume amplitudes. In certain embodiments, the audio feature 84 may be customized or configured to emit different types of alarms to distinguish between different functions, so that an operator or the patient 30 can distinguish between the sounds. For example, the audio feature 84 may emit different sounds for informing an operator about the status of wireless connectivity than for locating/identifying the monitor 12 or the sensor 14. The visual feature 86 (e.g., visual indicator 86) may be any indicator type that visually facilitates the identification of the audio/visual features 80, 81 by the operator or the patient 30. For example, the visual feature 86 may be indicator lights (e.g., flashing LEDs) or display screen graphics (e.g., E-Ink display). In some cases, the visual feature 86 may provide different patterns of flashing lights to inform the operator about the status of wireless connectivity than for locating/identifying the monitor 12 or the sensor 14. For example, the visual feature 86 may blink repeatedly at a set interval time when attempting to establish wireless connectivity between the monitor 12 and the sensor 14. In particular, the activation features 82, 83 may use the identification channel 90 to communicate with the audio feature 84, the visual feature 86, or a combination of the both (e.g., the audio/visual features 80, 81).

In certain embodiments, the activation feature 83 on the patient monitor 12 may be used to trigger the audio/visual feature 80 (e.g., the audio feature 84, the visual feature 86, or both) on the wireless sensor 14. For example, it may be desirable for an operator near the monitor 12 to locate and/or identify the sensor 14 coupled (e.g., paired) with the monitor 12. In such circumstances, an operator may engage the activation feature 83 on the patient monitor 12, which in turn may trigger the audio/visual feature 80 on the wireless sensor 14. In particular, the activation feature 83 on the monitor 12 triggers the audio/visual feature 80 on the sensor 14 through the identification channel 90. Similarly, it may be desirable for an operator near the sensor 14 to locate and/or identify the monitor 12 coupled (e.g., paired) with the sensor 14. In such circumstances, the activation feature 82 on the wireless sensor 14 may be used to trigger the audio/visual feature 81 on the patient monitor 12 through the identification channel 90. In other embodiments, two or more sensors 14 may be communicatively coupled to a single monitor 12 through the identification channel 90. Likewise, two or more monitors 12 may be communicatively coupled to a single sensor 14 through the identification channel 90. In such situations, the activation features 82, 83 provides a means for locating or identifying the desired device by triggering the audio/visual features 80, 81 on all the devices communicatively coupled through the identification channel 90. For example, for a monitor 12 coupled to two sensors 14, engaging the activation feature 81 on the monitor 12 may trigger the audio/visual features 80 on both sensors 14.

In yet other embodiments, the activation feature 83 on the patient monitor 12 may be used to identify or locate all the audio/visual features 80 on the wireless sensors 14 within a certain range. For example, engaging the activation feature 83 on the patient monitor 12 may trigger all the audio/visual features 80 within approximately 30 meters to 50 meters of the monitor 12, or within the potential range of wireless transmission for all the sensors 14 communicatively coupled to the monitor 12. Likewise, engaging the activation feature 82 on the wireless sensor 14 may trigger the audio/visual feature 81 on all the monitors 12 communicatively coupled to the sensors 14 within a particular range. In yet other embodiments, the activation features 82, 83 may be used to locate the closest audio/visual feature 80, 81 on either the monitor 12 or the sensor 14. In yet other embodiments, the wireless sensors 14 or the patient monitors 12 may include or enable a global positioning system (GPS). The activation feature 81 on the monitor 12 may trigger the operation of the GPS within the sensor 14, and may relay the position or coordinate information back to the monitor 12. In such embodiments, the audio/visual feature 80 on the sensor 14 may indicate that the monitor 12 is requesting GPS information through the audio feature 84 or the visual feature 86 (e.g., E-ink visual display feature).

FIG. 4 depicts a process flow diagram of an embodiment of a method 100 for identifying and/or locating the patient monitor 12 or the wireless sensor 14 with the audio/visual features 80, 81. To facilitate description of certain of the steps included in the method 100, reference is also made to FIG. 3, which schematically depicts the configuration obtained from performing the steps of the method 100. The method 100, in the embodiment of FIG. 4, includes establishing wireless communication between the patient monitor 12 and the wireless sensor 14 (block 102). As an example, wireless communication may be established through the identification channel 90 or the communication channel 92. The identification channel 90 may be used to exchange identification information (e.g., for the patient 30, the patient monitor 12, or the sensor 14) to couple or pair the monitor 12 with the sensor 14. In addition, the identification channel 90 may provide a communication pathway for the activation features 82, 83 to interact with the audio/visual features 80, 81, so as to provide a means for identifying and/or locating the coupled monitor 12 and sensor 14. The communication channel 92 may be used to exchange patient-specific data, such as physiological parameter trend data acquired over time for a particular patient 30. In certain embodiments, wireless communication between the sensor 14 and the monitor 12 (or between the activation features 82, 83 and the audio/visual features 80, 81) may occur through a single channel that may exchange both identification information and/or patient-specific data.

After wireless communication, and a coupling, is established between the patient monitor 12 and the sensor 14, an operator may engage the activation features 82 and 83 on the sensor 14 and the monitor 12, respectively (block 104). Engaging the activation features 82 and 83 may trigger the audio/visual features 80 and 81 (block 106). More specifically, an operator with access to the monitor 12 attempting to locate and/or identify the sensor 14 coupled (e.g., paired) with the monitor 12 may engage the activation feature 83 on the patient monitor 12, which in turn may trigger the audio/visual feature 80 on the sensor 14. Likewise, an operator with access to the sensor 14 attempting to locate and/or identify the monitor 12 coupled (e.g., paired) with the sensor 14 may engage the activation feature 82 on the sensor 14, which in turn may trigger the audio/visual feature 81 on the monitor 12.

The triggered audio/visual features 80, 81 may assist the operator or the patient 30 quickly and safely identify or locate the coupling or pairing between the patient monitor 12 and the wireless sensor 14 (block 108). A triggered audio/visual feature 80, 81 on either the monitor 12 or the sensor 14 may turn on the audio feature 84 or the visual feature 86. More specifically, the activated audio feature 84 (e.g., audio indicator 84) may be a speaker for emitting audible indications (e.g., alarms or beep tones), possibly with various frequencies, pitches, and/or volume amplitudes. The activated visual feature 86 (e.g., visual indicator 86) may be indicator lights (e.g., flashing LEDs) or display screen graphics (e.g., visual display feature). Accordingly, with multiple wireless sensors in use within a facility, an operator may efficiently and safely locate and/or identify through the audio feature 84, the visual feature 86, or both (e.g., audio/visual features 80, 81) the sensor 14 sending a signal, corresponding to a physiological measurement, to the monitor 12.

As such, in certain embodiments, the method 100 includes operating the wireless medical sensor 14 by wirelessly transmitting, via the wireless module 18, a signal from the patient monitor 12 to locate and/or identify the wireless medical sensor 14 in wireless communication with the patient monitor 12. The method 100 also includes receiving the signal at the wireless module 22 of the wireless medical sensor 14, and providing, in response to the signal, a user perceptible indication (e.g., audio/visual features 80, 81) on the wireless medical sensor 14.

FIG. 5 illustrates an embodiment of the patient monitoring system 10 having the wireless module 18 of the patient monitor 12 coupled to the wireless module 22 of the wireless sensor 14 through a pairing feature 110. As discussed in FIGS. 3 and 4, the monitor 12 and the sensor 14 may be coupled, or recoupled, using one of any number of wireless communication methods described above. For example, as described above, the wireless modules 18, 22 may be capable of communicating using the IEEE 802.15.4 standard (e.g., ZigBee, WirelessHART, or MiWi modules), the Bluetooth standard, the IEEE 802.11 standards, the ultra-wideband (UWB) standard, or the near-field communication (NFC) standard. In certain embodiments, such as in the illustrated embodiment, the wireless modules 18, 22 may be coupled through the NFC standard with the pairing feature 110 located on the wireless sensor 14. The pairing feature 110 on the sensor 14 may establish wireless communication with the monitor 12 upon physical contact between the pairing feature 110 and a identifier 112 on the patient monitor 12. In other embodiments, the pairing feature 110 on the sensor 14 may establish wireless communication with the monitor 12 when the pairing feature 110 is brought within close proximity of (e.g., near or within the vicinity of) the monitor 12 (e.g., the identifier 112 on the monitor). Upon successful pairing of the monitor 12 and the sensor 14, the activation features 82, 83 may be used to trigger the audio/visual features 80, 81 to identify and/or locate the coupled monitor 12 and sensor 14.

In particular, the pairing feature 110 on the wireless sensor 14 may be electrical circuitry or physical features located on the wireless sensor 14. Indeed, the pairing feature 110 may be any embedded or protruding feature on the sensor 14 that may enable the sensor 14 to recognize the sensor 14 and identify the proximity of the sensor 14 to the monitor 12. For example, the pairing feature 110 may be a NFC chip having electrical circuitry that generates a weak magnetic field upon activation by a switch or button 32. When the weak magnetic field generated by the pairing feature 110 is brought near (e.g., close proximity or physical contact) to the identifier 112 of the monitor 12, the weak magnetic may induce a magnetic field within the identifier 112 to create a radio field (e.g., radio waves or frequencies). The chip, or pairing feature 110, decodes the radio field to identify the monitor 12 and establishes one or more wireless communications channels. For example, as described above, the identification channel 90 may be established through NFC to exchange identification information between the wireless sensor 14 and the patient monitor 12 for the patient 30, for the patient monitor 12, or for the wireless sensor 14. In some situations, upon establishing the identification channel 90 through NFC, the communication channel 92 may be established though any of the other means of wireless communications described above, and may be used to exchange patient-specific data, such as physiological parameter trend data acquired over time for the patient 30.

In other embodiments, the pairing feature 110 may be polarized magnets embedded into the body 28 of the sensor 14. When the polarized magnets are brought near (e.g., close proximity or physical contact) to the identifier 112, the identifier 112 may recognize the magnets and establishes one or more wireless communications channels, as described above. In other embodiments, the pairing feature 110 may be one or more small protruding features having any geometric shape, such as spikes, bumps, etc. In such embodiments, the identifier 112 may have slots to insert the pairing feature 110, upon which the identifier 112 may recognize and establish one or more wireless communications channels, as described above.

In certain embodiments, two or more sensors 14 may be communicatively coupled to a single monitor 12 with the pairing feature 110. Likewise, two or more monitors 12 may be communicatively coupled to a single sensor 14 with the pairing feature 110. For example, upon successful pairing of one sensor 14 with a particular monitor 12 as described above, another sensor 14 with a corresponding pairing feature 110 may be coupled to the same monitor 12. In other embodiments, before a wireless channel is established between the sensor 14 and the monitor 12 (i.e., before the sensor 14 and the monitor 12 are coupled), the monitor 12 may check the functionality of the sensor 14 to ensure that the sensor 14 is in proper working condition. For example, the functionality check may include checking the emitter 24 and the detector 26 to ensure that they are working properly. As a further example, the functionality check may include checking the battery life or the memory storage space of the sensor 14 to ensure that the sensor 14 may continue to function properly within the near future. In certain embodiments, a failed functionality check may prevent the coupling of the sensor 14 with the monitor 12, and one or more audio/visual features 80, 81 may be used to alert the operator that the sensor 14 and the monitor 12 were unable to be coupled.

In particular, as described above with respect to FIGS. 3 and 4, upon successful pairing of the sensor 14 and the monitor 12, the activation features 82, 83 on the monitor 12 and the sensor 14 may trigger the audio/visual features 80, 81 (e.g., the audio feature 84, the visual feature 86, or both) on the sensor 14. Triggering the audio/visual features 80, 81 enable an operator to locate and/or identify one or more components within the system as described above.

In this manner, the sensor 14 and the monitor 12 may be coupled (i.e., paired), or recoupled, depending on the availability of the sensors 14 and the monitor 12 around the patient 30 within the facility. In particular, enabling the coupling and recoupling of wireless sensors 14 with patient monitors 12 provides the sensor 14 with greater flexibility and mobility within the facility.

FIG. 6 depicts a process flow diagram of an embodiment of a method 120 for coupling (i.e., pairing) one or more wireless sensors 14 to the patient monitor 12 with the pairing feature 110. To facilitate description of certain of the steps included in the method 120, reference is also made to FIG. 5, which schematically depicts the configuration obtained from performing the steps of the method 120.

The method 120, in the embodiment of FIG. 6, includes providing the sensor 14 having one or more pairing features 110 to be coupled to the monitor 12 (block 122). In particular, the pairing feature 110 on the sensor 14 is brought within close proximity (e.g., near) the identifier 112 on the monitor 12 (block 124). For example, close proximity may be approximately between 5 to 10 centimeters, 10 to 20 centimeters, 20 to 40 centimeters, and so forth. In other examples, the pairing feature 110 on the sensor 14 may physically contact the identifier 112 on the monitor 12. In yet other embodiments, the pairing feature 110 may be one or more small protrusions on the body 28 of the sensor 14 that are inserted into slots on the monitor 12. Upon successful identification of the pairing feature 110 and the sensor 14, one or more wireless communications channels are established as described above in FIG. 3. For example, the identification channel 90 may be established to couple the sensor 14 and the monitor 12 by exchanging identification information between the wireless sensor 14 and the patient monitor 12 for the patient 30, for the patient monitor 12, or for the wireless sensor 14.

In certain embodiments, the method 120 includes alerting an operator if the wireless module 18 of the patient monitor 12 is unable to couple to the wireless module 22 of the wireless sensor 14 through a pairing feature 110 (block 128). In such embodiments, the audio/visual features 80, 81 on the sensor 14 and the monitor 12 may be used to indicate that the sensor 14 and the monitor 12 are unable to be coupled. In addition, before a wireless channel is established between the sensor 14 and the monitor 12 (i.e., before the sensor 14 and the monitor 12 are coupled), the monitor 12 may check the functionality of the sensor 14 to ensure that the sensor 14 is in proper working condition. In certain embodiments, a failed functionality check may block the coupling of the sensor 14 with the monitor 12, and one or more audio/visual features 80, 81 may be used to alert the operator that the sensor 14 and the monitor 12 were unable to be coupled.

In other embodiments, two or more sensors 14, each with the pairing feature 110, may be provided and may be coupled to the monitor 12 (block 130). In particular, each pairing feature 110 may utilize one or more steps of the process 120, as described herein. In the illustrated embodiment, one or more sensors 14 with the pairing features 110 are provided and brought in proximity (e.g., near) to the identifier 122 on the monitor 12 before wireless communication, and coupling, is established. In other embodiments, one or more monitors 12 may be provided with the pairing features 110.

FIG. 7 illustrates an embodiment of the patient monitoring system 10 having the wireless module 18 of the patient monitor 12 coupled to the wireless module 22 of the wireless sensor 14 through one or more unique tokens 140. As discussed in FIGS. 3 and 4, the monitor 12 and the sensor 14 may be coupled, or recoupled, using one of any number of wireless communication methods described above. In certain embodiments, such as the embodiments illustrated in FIGS. 4 and 5, the wireless modules 18, 22 may be coupled through the NFC standard with the pairing feature 110 located on the wireless sensor 14. In the illustrated embodiment, the one or more tokens 140 may be used to couple the sensor 14 and the monitor 12, and to establish and/or initiate the wireless communications between the wireless modules 18, 22 of the sensor 14 and the monitor 12. In particular, in certain embodiments, a unique identifier may be provided on each of the one or more tokens 140. In certain embodiments, the unique identifier may be reasonably unique, such that it may not be universally unique, but may be unique enough to distinguish between devices within a particular facility or facilities. In such embodiments, the identifier 112 on the monitor 12, and a scanner 142 on the sensor 14, may identify and recognize the unique identifier on each of the tokens 140. For example, the identifier 112 or the scanner 142 may be any form of scanning device, such as a RFID scanner, a barcode scanner, a hole pattern identifier, a QR code scanner, or a combination thereof. The sensor 14 and the monitor 12 may scan for the corresponding unique identifier using any of the wireless methods provided above. In particular, upon successfully recognizing a matching pair (or pairs) of unique identifiers, the wireless modules 18, 22 of the monitor 12 and the sensor 14 may establish one or more wireless channels (e.g., identification channel 90 and/or communication channel 92) to transfer identification information and/or patient-specific information.

In certain embodiments, each token 140 may be split into one or more identical components, such that each component has the same unique identifier. For example, in the illustrated embodiment, the token 140 has a perforated edge 144 that may split or break the token 140 into two identical token components 146a and 146b. Each token component 146a and 146b has the same unique identifier 148 that is recognizable and/or identifiable by the monitor 12 or the sensor 14. For example, in the illustrated embodiment, the unique identifier 148 is a radio-frequency identification tag 150 (RFID tag). The RFID tag may be embedded into the token components 146a, 146b, or may be visible to the eye on the surface of the token components 146a, 146b. While the illustrated embodiment is representative of an active RFID tag 150, in other embodiments, the RFID tag 150 may be any form of RFID, such as a capactively coupled tag, an inductively coupled tag, a passive tag, a semi-active tag, or a combination thereof. FIGS. 8-10 illustrate other embodiments of the token 140 having other types of unique identifiers 148.

In particular, the token components 146a, 146b may be disposable tokens 140 that may be reused to couple one or more sensors 14 to one or more monitors 12 before they are disposed of. For example, token component 146b may be recognized and identified by the identifier 112 on the monitor 12 before being disposed of. Further, token component 146a may be recognized and identified by one or more sensors 14 before being disposed of. As such, the monitor 12 may be coupled to one or more sensors 14, as described above with respect to FIGS. 3-6. In other embodiments, both the sensor 14 and the monitor 12 may use a single token 140 to establish the wireless connection and pairing.

In addition, as described above with respect to FIGS. 3-6, upon successful pairing of the sensor 14 and the monitor 12, the activation features 82, 83 on the monitor 12 and the sensor 14 may trigger the audio/visual features 80, 81 (e.g., the audio feature 84, the visual feature 86, or both) on the sensor 14. Triggering the audio/visual features 80, 81 enable an operator to locate and/or identify one or more components within the system as described above.

FIG. 8 illustrates an embodiment of the token 140 having one or more hole patterns 152 representative of the unique identifier 148. In particular, the hole patterns 152 may be used for recognizing a matching pair (or pairs) of unique identifiers 148, so that the wireless modules 18, 22 of the monitor 12 and the sensor 14 may establish one or more wireless channels (e.g., identification channel 90 and/or communication channel 92) to transfer identification information and/or patient-specific information.

In particular, in the illustrated embodiment of the token 140 includes two token components 154a, 154b that are attached with the perforated edge 144. Each of the token components 154a, 154b have the same unique identifier 148 (e.g., hole pattern 152 or shape 153), and each component 154a, 154b may be inserted into a slot on either the monitor 14 or the sensor 12, such as within a slot 156 on the monitor 14. In such embodiments, the monitor 12 may register and translate the unique hole pattern 152 into a signal, and may scan for the corresponding unique signal using any of the wireless methods provided above. As indicated above, upon successfully recognizing a matching pair (or pairs) of unique identifiers, one or more wireless channels (e.g., identification channel 90 and/or communication channel 92) may be established to transfer identification information and/or patient-specific information, in accordance with the embodiments described herein.

FIG. 9 illustrates an embodiment of the token 140 having 2D or 3D quick response (QR) codes 158 representative of the unique identifier 148. In particular, the QR codes 158 may be used for recognizing a matching pair (or pairs) of unique identifiers 148, so that the wireless modules 18, 22 of the monitor 12 and the sensor 14 may establish one or more wireless channels (e.g., identification channel 90 and/or communication channel 92) to transfer identification information and/or patient-specific information.

In particular, the illustrated embodiment of the token 140 includes two token components 160a, 160b that are attached with the perforated edge 144. The token components 160a, 160b have the same unique identifier 148 (e.g., QR code 158) on each component which may be used for recognizing a matching pair (or pairs) of unique identifiers 148. The QR code 158 may be composed of black modules (square dots) arranged in a square grid on a white background. In particular, the QR code 158 may include different modes of information that may be decoded to provide identification information. For example, the information on the QR code 158 may be identification information for the sensor 14, the monitor 12, the patient 30, or a combination thereof. In certain embodiments, the QR codes 158 may be encrypted to ensure the protection of confidential information (e.g., such as confidential patient information). As described above, the unique identifier 148 may be recognized and identified by an appropriate identifier, such as the identifier 112 on the monitor 12 or the scanner 142 on the sensor 14. Upon successfully recognizing a matching pair (or pairs) of unique identifiers, one or more wireless channels (e.g., identification channel 90 and/or communication channel 92) may be established to transfer identification information and/or patient-specific information, in accordance with the embodiments described herein.

FIG. 10 illustrates an embodiment of the token 140 having bar codes 162 representative of the unique identifier 148. In particular, the bar codes 162 may be used for recognizing a matching pair (or pairs) of unique identifiers 148, so that the wireless modules 18, 22 of the monitor 12 and the sensor 14 may establish one or more wireless channels (e.g., identification channel 90 and/or communication channel 92) to transfer identification information and/or patient-specific information.

In particular, the illustrated embodiment of the token 140 includes three token components 164a, 164b, and 164c that are attached with two perforated edges 144. Each of the token components 164a, 164b, and 164c have the same unique identifier 148 (e.g., bar codes 162) on each component which may be used for recognizing a matching pair (or pairs) of unique identifiers 148. While the illustrated embodiment provides three token components 164a, 164b, and 164c with the same unique identifier 148, other embodiments may provide four, five, six, or more token components. In particular, the plurality of token components 164a, 164b, and 164c may be used to couple a plurality of sensors 14 with a plurality of monitors 12. As noted above, the token components may be used more than once, but may be made of disposable material to be discarded after use.

Moreover, as described above, the unique identifier 148 (e.g., bar codes 162) may be recognized and identified by an appropriate identifier, such as the identifier 112 on the monitor 12 or the scanner 142 on the sensor 14. Upon successfully recognizing a matching pair (or pairs) of unique identifiers, one or more wireless channels (e.g., identification channel 90 and/or communication channel 92) may be established to transfer identification information and/or patient-specific information, in accordance with the embodiments described herein.

FIG. 11 depicts a process flow diagram of an embodiment of a method 170 for coupling (i.e., pairing) one or more wireless sensors 14 to the patient monitor 12 with one or more unique tokens 140. To facilitate description of certain of the steps included in the method 170, reference is also made to FIGS. 7-10, which schematically depict the configuration obtained from performing the steps of the method 170.

The method 170, in the embodiment of FIG. 4, includes breaking the unique token 140 into one or more unique tokens 140, such as, for example, or one or more token components 146a, 146b, 154a, 154b, 160a, 160b, 164a, 164b, or 164c (block 172). In certain embodiments, the unique token 140 may be used to couple the sensor 14 and the monitor 12, and to establish the wireless communications between the wireless modules 18, 22 of the sensor 14 and the monitor 12. In particular, in certain embodiments, the unique token 140 may be split or torn into one or more token components (e.g., a first token and a second token), where each token component includes the same unique identifier 148. In other embodiments, the unique token 140 may not be torn into smaller components, and instead include a single unique identifier 148. The unique identifier may be reasonably unique, such that it may not be universally unique, but may be unique enough to distinguish between devices within a particular facility or facilities. As described above in FIGS. 7-10, the unique identifier 148 may be the RFID tag 150, the unique hole pattern 152 (e.g., hole cutout pattern 152), the QR code 158, the bar codes 162, or a combination thereof.

After breaking the unique token 140 into one or more token components (e.g., the first token and the second token), each token component may be scanned or recognized by the identifier 112, or the scanner 142. In such embodiments, the first token may be brought near (e.g., close proximity or physical contact) the identifier 112 on the monitor 12 (block 174). Similarly, the second token may be brought near (e.g., close proximity or physical contact) the scanner 142 on the sensor 14 (block 176). The identifier 112 on the monitor 12, and a scanner 142 on the sensor 14, may identify and recognize the unique identifier 148 on each of the tokens components, and may scan a particular region to find corresponding unique identifiers 148. Upon successfully recognizing a matching pair (or pairs) of unique identifiers 148, the wireless modules 18, 22 of the monitor 12 and the sensor 14 may establish one or more wireless channels (e.g., identification channel 90 and/or communication channel 92) to transfer identification information and/or patient-specific information (block 178), in accordance with the embodiments described herein.

In certain embodiments, each token component (e.g., the first token or the second token) may be reused to couple another sensor 14 or another monitor 12 to the system of coupled sensor 14 and monitor 12 (block 180). For example, the second token may be reused by bringing it near (e.g., close proximity or physical contact) to another sensor 174 to establish wireless communication between the second sensor 174 and the patient monitor 12. After using, or reusing, the token components, they may be disposed to prevent the accidental coupling of other sensors 14 to other monitors 12 (block 182).

FIG. 12 illustrates an embodiment of the visual feature 86 (i.e., a visual display) on the wireless sensor 14 that can be performed in conjunction with or independently of the systems and methods of FIGS. 3-11. In particular, the visual feature 86 may incorporate an electronic paper display 190 (e.g., E-ink display 190) on one or more types of sensors 14, such as the pulse oximetry sensor 192 described in FIGS. 1-11, or an INVOS sensor 194. The INVOS sensor 194 may be an INVOS® cerebral/somatic sensor available from Somanetics Corporation, and may be configured to perform regional oximetry. Indeed, the sensor 14 may be any form of wireless sensor 14 that is capable of establishing wireless communication with the patient monitor 12 using any suitable wireless standard, such as those described above.

As described herein, the INVOS sensor 194, illustrated as wirelessly connected to the monitor 12, may include a sensor body 196 that houses the emitter 198 for emitting light at certain wavelengths into a tissue of a patient and the detectors 200 for detecting the light after it is reflected and/or absorbed by the blood and/or tissue of the patient. The sensor body 196 may be formed from any suitable material, including rigid or conformable materials, such as fabric, paper, rubber or elastomeric compositions (including acrylic elastomers, polyimide, silicones, silicone rubber, celluloid, PMDS elastomer, polyurethane, polypropylene, acrylics, nitrile, PVC films, acetates, and latex). In particular, the sensor body 196 may be formed out of a plurality of laminated layers. Generally, the laminated layers may be provided to protect the emitter 198 and the detectors 200 from damage, and also to enhance patient comfort. Thus, any number of padding layers may be suitably provided depending on the desired end use of the sensor 194.

In particular, the INVOS sensor 194 may include a top layer 202 and a bottom layer 204. The bottom layer 204 may include a patient-contacting adhesive layer 206 laminated on the bottom layer 204, and may include any adhesive material suitable for integration into medical devices (e.g., a hypoallergenic adhesive material). In some embodiments, the adhesive material may be substantially transparent with respect to the wavelengths of light used for the oximetry measurements performed by the sensor 194. In certain embodiments, the top layer 202 may be the display layer, and may include the electronic paper display 190 (e.g., E-ink display 190). The top layer 202 may be configured to prevent the ingress of light, which may interfere with oximetry measurements, into the INVOS sensor 194. Indeed, an opaque paint, thin polymeric layer, or similar covering may be applied to the top layer 202 below the electronic paper display 190 to prevent the ingress of light into the INVOS sensor 194. Moreover, because the top layer 202 may be the outermost layer of the sensor 194, various indications may be provided thereon, such as decorative markings, placement instructions, trade names, indications for use (e.g., indications for adult or neonate use), and so forth within or around the electronic paper display 190.

The electronic paper display 190 may include several advantages when used in conjunction with medical sensors 14. For example, electronic paper displays 190 have a paper-like look that provides a high contrast, flicker-free display with a wide viewing angle and relative ease of readability under a wide range of lighting conditions, including low light. Because such electronic paper displays, including electrophoretic displays, are thin and relatively flexible, these displays may be incorporated into the sensors 14 that comfortably conform to a patient's tissue. An additional benefit provided by the sensors 14 that include electronic paper may be reduced power consumption because electronic paper displays 190 only consume power when new information is being written to the display, i.e., power is not consumed to maintain information on the display 190. For sensors 14 that operate remotely, such reduced power consumption may lead to increased wear times and decreased battery waste, as the batteries may be recharged less frequently. In addition, electronic paper displays 190, because they have relatively low power consumption, may not experience substantial temperature increases during operation, and may be more comfortable for the wearer.

In certain embodiments, the visual feature 86 may be the electronic paper display 190 on the wireless sensors 14. As described above, the audio/visual features 80, 81 of the sensors 14 may be used to indicate the coupling status between the monitor 12 and the sensor 14. For example, the audio/visual features 80, 81 may alert an operator when the sensor 14 and the monitor 12 are coupled, are unable to be coupled, or when wireless connectivity is lost. In other embodiments, the audio/visual features 80, 81 may locate and/or identify one or more wireless sensors 14 coupled to the patient monitor 12, or vise versa. For example, the visual feature 86 (e.g., electronic paper display 190) may display the connectivity status of the sensor 14 and the monitor 12, may display warnings to the operator when the sensor 14 is coupled (or not coupled) to the monitor 12, may display warnings to the operator when wireless connectivity is lost between the sensor 14 and the monitor 12, or may display information to help identify and/or locate one or more wireless sensors 14 coupled to the patient monitor 12, or vise versa. In certain embodiments, the electronic paper display 190 may include a timer that may clear the electronic paper display 190 after a set amount of time of displaying the physiological parameter or patient related information. For example, after a period of inactivity (e.g., approximately 10 minutes to 20 minutes, 40 minutes to 60 minutes, 60 minutes or more), the electronic paper display 190 may clear to protect the patient identification information.

In particular, the electronic paper display 190 may display patient-specific data, such as physiological parameter trend data acquired over time for the patient 30. For example, the sensor 14 may be attached to the finger of the patient 30, and may be configured to store patient-specific data (e.g., physiologic trend data acquired over time for a particular patient) in the access memory (RAM) 60 and/or non-volatile (NV) memory 62. Such data may also be transferred to the patient monitor 12 through the communication channel 92. Such data may also be displayed on the electronic paper display 190 for the operator to view and assess. In particular, displaying the physiological data on the electronic paper display 190 may enable an operator to immediately assess the measured physiological parameters of the patient 30, even when the patient monitor 12 is not locally available. In other embodiments, it may be beneficial to use the audio feature 84 of the audio/visual features 80, 81 to alert the operator. For example, the visual feature 86 on the INVOS sensor 194 may be a color changing boundary 208 (e.g., color changing band 208). In particular, the color changing boundary 208 may change into a plurality of colors to alert the operator or to help identify and/or locate the sensor 14 when the activation feature 83 is engaged on the monitor 12.

As illustrated, and as described above, the wireless module 18 of the patient monitor 12 establishes the identification channel 90 and the communication channel 92 with the wireless module 22 of the sensors 14 (e.g., pulse oximetry sensor 192 and INVOS sensor 194). As noted above, the wireless module 22 of the sensors 14 may establish the wireless communication (e.g., identification channel 90 and/or communication channel 92) with the wireless module 18 of the patient monitor 12 using any suitable protocol. Again, the identification information on the display feature may indicate the coupling status of the wireless sensors 14 and patient monitors 12, while the measured physiological parameter information may enable an operator to remotely assess the patient's condition. In other embodiments, a transmitting device 210 carrying identification information may be used to establish the identification channel 90 with the sensors 14. For example, the transmitting device 210 may be an electronic device (e.g., smart phone, handheld computer, tablet computer, laptop computer, patient monitor 12 etc.), or may be any electronic device that carries patient identification information.

FIG. 13 depicts a process flow diagram of an embodiment of a method 220 for displaying on a visual feature 86 (e.g., electronic paper or electronic ink display 190) patient-specific physiological information and/or patient identification information transferred from the transmitting device 210 to the wireless sensors 14. To facilitate description of certain of the steps included in the method 220, reference is also made to FIG. 12, which schematically depicts the configuration obtained from performing the steps of the method 220.

The method 220, in the embodiment of FIG. 13, includes establishing wireless communication between the transmitting device 210 and the wireless sensor 14 (block 222). In certain embodiments, the transmitting device 210 may be the monitor 12 or a personal mobile device (e.g., smart phone, handheld computer, tablet computer, etc.). As an example, wireless communication may be established through the identification channel 90 or the communication channel 92.

In certain embodiments, patient-specific physiological information and/or identification information (e.g., patient identification information) may be transferred between the transmitting device 210 (e.g., electronic device) and the sensor 14 (block 224). In particular, the identification channel 90 may be used to exchange identification information (e.g., for the patient 30, the patient monitor 12, or the sensor 14) between the transmitting device 210 and the sensor 14, and to couple or pair the monitor 12 with the sensor 14. In addition, the identification channel 90 may provide a communication pathway for the activation features 82, 83 to interact with the audio/visual features 80, 81, so as to provide a means for identifying and/or locating the coupled monitor 12 and sensor 14. The communication channel 92 may be used to exchange patient-specific data, such as physiological parameter trend data acquired over time for a particular patient 30. In certain embodiments, wireless communication between the sensor 14 and the monitor 12 (or between the activation features 82, 83 and the audio/visual features 80, 81) may occur through a single channel that may exchange both identification information and/or patient-specific data.

In certain embodiments, the audio/visual features 80, 82 may include the visual feature 86, which may be a visual display, such as the electronic ink display 190. In certain embodiments, physiological parameters and/or patient identification information may be displayed on the electronic paper display 190. The electronic paper display 190 may be one or more of an electrophoretic display, an electronic ink display, an electro-wetting display, a bistable liquid crystal display, or a cholesteric liquid crystal display. The transferred information from the transmitting device 210 to the sensors 14 may be displayed on the visual feature 86 (block 226).

What is claimed is:

1. A patient monitoring system comprising:
a wireless medical sensor; and
a patient monitor in wireless communication with the wireless medical sensor, the patient monitor comprising:
an activation feature configured to be activated via a user;
a processor configured to generate a signal to locate or identify the wireless medical sensor in response to activation of the activation feature; and
a first wireless module configured to wirelessly communicate with the wireless medical sensor and to transmit the signal to the wireless medical sensor,
wherein the wireless medical sensor is configured to measure a physiological parameter of a patient, the wireless medical sensor comprising:
a second wireless module configured to wirelessly communicate with the patient monitor and to receive the signal; and
an indicator feature configured to provide a user perceptible indication in response to the signal.

2. The patient monitoring system of claim 1, wherein the wireless medical sensor comprises a pulse oximetry sensor.

3. The patient monitoring system of claim 1, wherein the user perceptible indication comprises an audible indication, a visual indication, or both.

4. The patient monitoring system of claim 1, wherein the indicator feature comprises a speaker configured to emit an audible indication in response to the signal.

5. The patient monitoring system of claim 1, wherein the indicator feature comprises a light emitting diode configured to emit light in response to the signal.

6. The patient monitoring system of claim 1, wherein the indicator feature comprises a display configured to provide a visual indication in response to the signal.

7. The patient monitoring system of claim 6, wherein the display comprises an electronic paper display.

8. The patient monitoring system of claim 7, wherein the electronic paper display comprises a band that changes color in response to the signal.

9. The patient monitoring system of claim 7, wherein the electronic paper display is configured to display identification information received from the patient monitor, and wherein the identification information comprises a patient ID number, a patient name, a unique bar code number, a unique serial number, a patient identification tag or bracelet, or a combination thereof.

10. The patient monitoring system of claim 7, wherein the electronic paper display is configured to display the physiological parameter.

11. The patient monitoring system of claim 7, wherein the display comprises an electronic paper display, and wherein the electronic paper display is configured to display identification information received from the patient monitor.

12. A wireless medical sensor, comprising:
at least one sensing element configured to measure a physiological parameter of a patient;
a wireless module configured to wirelessly communicate with a patient monitor and to wirelessly receive an identification signal from the patient monitor; and
an indicator feature configured to provide a user perceptible indication identifying the wireless medical sensor, in response to the identification signal.

13. A patient monitoring system comprising:
a wireless medical sensor configured to obtain a signal related to a physiological parameter from a patient, the wireless medical sensor comprising:
a first wireless module configured to wirelessly transmit and receive data;
an electronic paper display configured to display the physiological parameter and patient related information; and
an electronic device, separate from the wireless medical sensor, configured to wirelessly transmit patient related information to the wireless medical sensor via a second wireless module upon establishing communication with the wireless medical sensor.

14. The patient monitoring system of claim 13, wherein the wireless medical sensor comprises a pulse oximetry sensor or a regional oximetry sensor.

15. The patient monitoring system of claim 13, wherein the wireless medical sensor comprises a processor configured to process the signal to obtain the physiological parameter and to update the display of the physiological parameter on a periodic basis.

16. The patient monitoring system of claim 13, wherein the electronic paper display comprises one or more of an electrophoretic display, an electronic ink display, an electro-wetting display, a bistable liquid crystal display, or a cholesteric liquid crystal display.

17. The patient monitoring system of claim 13, wherein the electronic paper display is configured to consume power only upon updating the display of the physiological parameter or patient related information.

18. A wireless medical sensor, comprising:
at least one sensing element configured to obtain a signal related to a physiological parameter from a patient;
a wireless module configured to wirelessly transmit and receive data; and
an electronic paper display configured to display the physiological parameter and patient related information.

19. A patient monitoring system comprising:
a wireless medical sensor configured to obtain a signal related to a physiological parameter from a patient, the wireless medical sensor comprising a first wireless module configured to wirelessly transmit and receive data;
a patient monitor comprising a second wireless module, wherein the second wireless module is configured to wirelessly communicate with the wireless medical sensor upon being operatively paired with the wireless medical sensor; and
a pairing feature configured to operatively pair the wireless medical sensor and the patient monitor to enable the wireless communication.

20. The patient monitoring system of claim 19, wherein the pairing feature comprises a token having a unique identifier to operatively pair the wireless medical sensor and the patient monitor.

21. The patient monitoring system of claim 20, wherein the token comprises a first token component configured to be disposed in the wireless medical sensor, and a second token component configured to be disposed in the patient monitor.

22. The patient monitoring system of claim 19, wherein the wireless medical sensor comprises a sensor body, and the pairing feature comprises a plurality of protruding features extending from the sensor body.

23. The patient monitoring system of claim 19, wherein the wireless medical sensor comprises a sensor body, and the pairing feature comprises one or more polarized magnets embedded in the sensor body.

24. The patient monitoring system of claim 19, wherein the wireless medical sensor comprises a sensor body, the pairing feature comprises electrical circuitry embedded in the sensor body, and the electrical circuitry is configured to produce a magnetic field.

25. The patient monitoring system of claim 19, wherein the wireless medical sensor comprises the pairing feature, the patient monitor comprises an additional pairing feature, and the pairing feature and the additional pairing feature are configured to operatively pair the wireless medical sensor and the patient monitor upon physical contact between the pairing feature and the additional pairing feature.

26. The patient monitoring system of claim 25, wherein the pairing feature comprises electrical circuitry, polarized magnets, or a plurality of protruding features, and the additional pairing feature comprises a near field communications chip, a scanner, or a slot.

27. The patient monitoring system of claim 19, wherein the pairing feature comprises a first pairing component on the wireless sensor and a second pairing component on the patient monitor, and wherein the first and second pairing components are configured to establish a wireless communication channel upon physical contact with each other.

28. A patient monitoring system comprising:
a wireless medical sensor configured to obtain a signal related to a physiological parameter from a patient, the wireless medical sensor comprising a first wireless module configured to wirelessly transmit and receive data;
a patient monitor configured to wirelessly transmit to and receive data from the wireless medical sensor via a second wireless module; and
one or more tokens having a unique identifier, wherein the one or more tokens are configured to initiate wireless communication between the wireless medical sensor and the patient monitor based at least in part on the unique identifier.

29. The patient monitoring system of claim 28, wherein the unique identifier comprises a RFID tag, a 3D quick response (QR) code, a 2D QR code, a unique hole pattern, a unique barcode, or a combination thereof.

30. The patient monitoring system of claim 28, wherein at least one of the one or more tokens is configured to be separated into a first component and a second component, and wherein the first component is configured to be disposed in the wireless medical sensor, and the second component is configured to be disposed in the patient monitor.
